(19) United States
(12) Patent Application Publication
(10) Pub. No.: US 2026/0345662 A1
(43) Pub. Date: Nov. 5, 2026

(54) METHODS AND COMPOSITIONS TO TREAT OBESITY BY TARGETING KCC2 AND NKCC1

(71) Applicant: THE JACKSON LABORATORY, Bar Harbor, ME (US)

(72) Inventors: Kristen O'CONNELL, Bar Harbor, ME (US); Catherine KACZOROWSKI, Ann Arbor, MI (US)

(21) Appl. No.: 19/487,357

(22) PCT Filed: May 24, 2024
(86) PCT No.: PCT/US2024/031052

§ 371 Date: Nov. 24, 2025

Related U.S. Application Data
(60) Provisional application No. 63/504,429, filed on May 25, 2023.

(51) Int. Cl. A61K 31/7105; A61K 31/192; A61K 38/46; A61P 3/04

(52) U.S. Cl. CPC A61K 31/7105; A61K 31/192; A61K 38/465; A61P 3/04

(57) ABSTRACT

This disclosure provides methods for treating obesity in a subject with an agent that inhibits Na-K-Cl co-transporter 1 (NKCC1) and/or an agent that increases K-Cl co-transporter 2 (KCC2); and a pharmaceutical composition to treat same containing each or both of these agents.

### STATEMENT REGARDING FEDERALLY SPONSORED RESEARCH

This invention was made with government support under R01 DK102918 awarded by the National Institutes of Health. The government has certain rights in the invention.

### BACKGROUND OF THE DISCLOSURE

Obesity has been a significant public health concern for 50 years and is a significant comorbidity for diseases such as type 2 diabetes, cardiovascular disease, cancer, Alzheimer's disease, and COVID-19. Diet and lifestyle interventions are generally effective for weight loss, but long-term adherence is generally low and most individuals regain the lost weight within 5 years (Berk et al., 2018, Diabetologia 61:790-799; Fothergill et al., 2016, Obesity (Silver Spring) 24:1612-1619; Greenway, 2015, Int J Obes 39:1188-1196; Hall and Guo, 2017, Gastroenterology 152:1718-1727 e1713; Sumithran and Proietto, 2013, Clin Sci (Lond) 124:231-241). Experimental evidence indicates that high-fat, calorie-dense diets cause remodeling of neurons and circuits governing energy balance, highlighting an important role for central nervous system (CNS) plasticity in the development and maintenance of obesity (Baver et al., 2014, J Neurosci 34:5486-5496; Wei et al., 2015, Sci Rep 5:16810; Beutler et al., 2020, Elife 9: e55909; Mazzone et al., 2020, Nat Neurosci. 23:1253-1266; Rossi et al., 2019, Science 364:1271-1274).

However, an understanding of how diet and obesity alter CNS function remains relatively poor; and thus, no treatment targeting the CNS has been developed. Thus there remains a need in the art for pharmaceutical agents and therapeutic methods for treating or preventing obesity.

### SUMMARY

Provided herein are pharmaceutical agents and therapeutic methods for treating or preventing obesity based on alleviating or modulating neuronal dysfunction in obesity aimed at novel targets for treating this disease.

Described herein are methods for treating obesity in a subject comprising administering an agent to increase function or expression of K-Cl co-transporter 2 protein (KCC2) in CNS AgRP-expressing neurons. In some embodiments, the agent can be capable of increasing expression of Slc12a5 gene encoding KCC2, wherein inter alia such agent can be CRISPR activation-Cas9 complex with gRNA that targets Slc12a5 gene.

In another aspect, the disclosure provides pharmaceutical compositions for treating obesity that comprises a chemical compound that increases KCC2 function or expression, and a pharmaceutical acceptable carrier.

Also described herein are methods for treating obesity in a subject comprising administering an agent to decrease function or expression of Na-K-Cl co-transporter 1 protein (NKCC1) in CNS AgRP-expressing neurons. In some embodiments, the agent can be a chemical compound, including but not limited to bumetanide, azosemide, DIMAEB, BUM66, or a combination thereof. In some embodiments, the agent can be capable of inhibiting expression of Slc12a2 gene encoding NKCC1, such agent can be a CRISPR-Cas9 complex with gRNA that targets Slc12a2 gene.

In another aspect, the disclosure provides pharmaceutical compositions for treating obesity comprising a chemical compound that inhibits NKCC1 function or expression, and a pharmaceutical acceptable carrier. In some embodiments, such compounds are bumetanide, azosemide, DIMAEB, BUM66, or combinations thereof.

In another aspect, the disclosure provides a pharmaceutical composition to treat obesity comprising a first compound that inhibits NKCC1 function or expression, a second chemical compound that increases KCC2 function or expression, and a pharmaceutical acceptable carrier. In some embodiments, the first compound is bumetanide, azosemide, DIMAEB, BUM66, or a combination thereof..

These and other features, objects, and advantages of this invention will become better understood from the description that follows. In the description, reference was made to the accompanying drawings, which form a part hereof and in which there was shown by way of illustration, not limitation, embodiments of the invention. The description of preferred embodiments was not intended to limit the invention to cover all modifications, equivalents, and alternatives. Reference should therefore be made to the claims recited herein for interpreting the scope of the invention.

### BRIEF DESCRIPTION OF THE DRAWINGS

The disclosure will be better understood and features, aspects, and advantages other than those set forth above will become apparent when consideration was given to the following detailed description thereof. Such detailed description refers to the following drawings.

FIG. 1A through FIG. 1M show that female mice are resistant to diet-induced changes in agouti-related protein (AgRP) neuronal hyperexcitability. FIG. 1A shows bodyweight curves for male mice during 8 weeks of high fat diet (HFD) feeding. FIG. 1B shows bodyweight curves for female mice during 8 weeks of HFD feeding. FIG. 1C shows representative traces of AgRP neurons from female mice fed a normal calorie diet (NCD_{fed})(top), NCD_{fast} (middle), or HFD_{8wk} in fed state (bottom; Calibration: 1s, 5 mV). FIG. 1D shows that HFD_{8wk}, but not fasting, significantly increased AgRP neuronal firing rate. FIG. 1E shows that HFD_{8wk}, but not fasting, significantly depolarized neuronal resting membrane potential (RMP). FIG. 1F shows representative traces of AgRP leptin inhibition from female NCD_{fast} (top) or HFD_{8wks} (bottom) mice (Calibration: 2s, 10 mV). FIG. 1G shows summary response of AgRP neurons to leptin. FIG. 1H shows bodyweight curves for female mice during 24 weeks of HFD feeding. FIG. 1I shows representative traces of AgRP baseline firing from aged female NCD_{fed} (top), NCD_{fast} (middle), or HFD_{24wk} (bottom) mice (bottom; Calibration: 1 s, 5 mV). FIG. 1J shows that fasting and HFD_{24wk} feeding significantly increased AgRP neuronal firing rate. FIG. 1K shows that only HFD_{24wk} hyperpolarized neuronal resting membrane potential (RMP). FIG. 1L shows representative traces of AgRP leptin inhibition from aged female NCD_{fast} (top) and HFD_{24wk} (bottom) (Calibration: 2 s, 10 mV). FIG. 1M shows summary response of AgRP neurons to leptin. For all analyses, diet conditions compared to NCD using standard ANOVA with post hoc Tukey's multiple comparisons test and a mixed-effects repeated measures ANOVA for repeated measures (*=p<0.05; **=p<0.01; ***=p<0.001, ****=p<0.0001) .

FIG. 2A shows representative traces of miniature excitatory synaptic currents (mEPSCs) in AgRP neurons from male mice fed NCD or HFD_{8wk} (Calibration: 2 s, 4 pA).

FIG. 2B shows representative traces of mEPSCs in AgRP neurons from female mice fed NCD or HFD_{8wk} (Calibration: 2 s, 4 pA). FIG. 2C shows mean mEPSC frequency was increased in fasted male mice. FIG. 2D shows mean mEPSC amplitude was decreased in AgRP neurons from fasted male mice compared to fed males. FIG. 2E shows mean mEPSC frequency in AgRP neurons from female mice was increased in fasted compared to fed females. FIG. 2F shows mean amplitude was not changed by diet or fasting in female mice. For all violin plots, dashed lines indicates median and dotted lines indicate quartiles. Statistical comparisons using two-way ANOVA with sex and diet as main effects; post hoc pairwise comparisons performed using Tukey's multiple comparisons test (*=p<0.05; **=p<0.01; ***=p<0.001, ****=p<0.0001).

FIG. 3A through FIG. 3F shows that long-term HFD feeding had a minimal impact on excitatory input but resulted in increased inhibitory input to AgRP neurons. FIG. 3A shows representative traces of miniature inhibitory synaptic currents (mIPSCs) in AgRP neurons from male mice fed NCD or HFD_{8wk} (Calibration: 1 s, 20 pA). FIG. 3B shows representative traces of mIPSCs in AgRP neurons from female mice fed NCD or HFD_{8wk} (Calibration: 1 s, 20 pA). FIG. 3C shows increased mean mIPSC frequency in AgRP neurons from HFD_{8w} male mice compared to NCD males. FIG. 3D shows increased mean mIPSC amplitude in AgRP neurons from HFD_{8w} male mice compared to NCD males. FIG. 3E shows decreased mean mIPSC frequency in AgRP neurons from NCD_{fasted} female mice compared to NCD_{fed}, HFD_{fed}, and HFD_{fasted} females. FIG. 3F shows increased mean amplitude in fasted HFD_{8w} female mice. For all violin plots, dashed line indicates medians and dotted lines indicate quartiles. Statistical comparisons using two-way ANOVA with sex and diet as main effects; post hoc pairwise comparisons performed using Tukey's multiple comparisons test (*=p<0.05; **=p<0.01; ***=p <0.001, ****=p<0.0001) .

FIG. 4A shows representative traces of the response to one second (2 s) puff of 10 μM glutamate in AgRP neurons from NCD (top) and HFD (bottom) AgRP neurons. FIG. 4B shows AgRP neuronal firing rates before (black dots) and after (blue dots) puff application of glutamate (NCD: n=12 neurons, HFD: n=12 neurons; repeated measures two-way ANOVA with Tukey's post hoc multiple comparisons test). FIG. 4C shows representative traces illustrating the response of AgRP neurons from NCD (top) and HFD_{8w} (bottom) mice to puff application of 100 μM GABA. FIG. 4D shows AgRP neuronal firing rates before (black dots) and after (blue dots) GABA application (NCD: n=12 neurons; HFD=12 neurons, repeated measures two-way ANOVA with Tukey's post hoc multiple comparisons test).

FIG. 5A through FIG. 5E show that long-term HFD feeding was associated with depolarization of E_{GABA}, resulting in a loss of GABA-mediated synaptic inhibition. FIG. 5A shows that cation channel rhodopsin 2 (ChR2) was expressed in presynaptic inhibitory Lepr+ neurons from the ventral dorsomedial hypothalamus (vDMH; top) (inset, terminals in red (right)). FIG. 5B shows representative traces from optogenetically inhibited AgRP neurons from NCD (black trace) and HFD_{8w} (blue trace) mice (Calibration 2 s, 5 mV). FIG. 5C shows changes in AgRP neuronal firing rate in response to light-evoked activation of GABAergic vDMH presynaptic terminals (Light-Baseline; paired t-test). FIG. 5D are representative I/V curves showing EG_{ABA} was depolarized by approximately +20 mV in HFD males compared to NCD (inset; representative traces from NCD (top left) and HFD_{8w} (bottom right) males. Red traces=−40 mV). FIG. 5E shows mean E_{GABA} in AgRP neurons from NCD (n=24 neurons), NCD_{fast} (n=7 neurons), HFD_{2d} (n=10 neurons), and HFD_{8w} (n=37 neurons) mice. Statistical comparisons using ordinary one-way ANOVA with Dunnett's post hoc multiple comparisons test. (*=p<0.05; **=p<0.01; ***=p<0.001, ****=p<0.0001).

FIG. 6A shows representative response to puff application of 100 μM GABA in HFD_{8w} male mice, following vehicle (artificial cerebrospinal fluid: aCSF) incubation and bumetanide incubation. FIG. 6B shows summary response of FIG. 6A following vehicle incubation and bumetanide incubation. FIG. 6C shows unilateral AAV-syn-FLEX-HA-KCC2/FLEX-GFP expression in the arcuate nucleus (ARC) of AgRP^{Cre/+} male mice. FIG. 6D shows that bodyweight was not altered 6-weeks post-surgery but was significantly increased in HFD_{fed} AgRP^{+/+} mice. FIG. 6E shows that AgRP^{cre/+} mice were not different from NCD_{fed} mice at 28-weeks of age (>10 weeks post-surgery), suggesting that KCC2 expression in the AgRP^{cre/+} mice prevented weight gain caused by HFD. FIG. 6F is a model illustrating diet-induced dyshomeostasis of intraneuronal Cl⁻ due to loss of KCC2 function and restoration of GABA-mediated inhibition following NKCC1 antagonism with bumetanide and KCC expression.

### DETAILED DESCRIPTION OF THE DISCLOSURE

Provided herein are pharmaceutical compositions and therapeutic methods for treating obesity by targeting Na-K-Cl co-transporter 1 (NKCC1) and K-Cl co-transporter 2(KCC2) to restore low intracellular Cl-concentration in agouti-related protein (AgRP) expressing neurons.

AgRP-expressing neurons integrate peripheral and central signals to modulate feeding and bodyweight (Morton and Schwartz, 2001, Int J Obes Relat Metab Disord 25(Suppl 5): S56-62; Könner et al., 2007, Cell Metab 5:438-449; Andrews et al., 2008,

Nature 454:846-851; Varela and Horvath, 2012, EMBO Rep 13: 1079-1086; Krashes et al., 2014, Nature 507: 238-242; Garfield et al., 2016, Nat Neurosci 19: 1628-1635). Activation of AgRP neurons has been associated with hunger and food seeking (van den Top et al., 2004, Nat Neurosci 7: 493-494; Takahashi and Cone, 2005, Endocrinology 146: 1043-1047; Baver et al., 2014, J Neurosci 34: 5486-5496; Mandelblat-Cerf et al., 2015, Elife 4: e07122). Stimulation drives voracious feeding (Aponte et al., 2011, Nat Neurosci 14, 351-355; Krashes et al., 2011, J Clin Invest 121: 1424-1428; Chen et al., 2016, Elife 5: e18640), while inhibition or ablation results in hypophagia or starvation (Krashes et al., 2011, J Clin Invest 121: 1424-1428; Luquet et al., 2005, Science 310:683-685). High-fat diet (HFD) consumption alters the function of AgRP neurons (Beutler et al., 2020, Elife 9: e55909; Mazzone et al., 2020, Nat Neurosci. 23(10): 1253-1266), resulting in persistent activation and resistance to modulation by physiological cues of hunger or satiety (Baver et al, 2014, J Neurosci 34: 5486-5496; Wei et al., 2015, Sci Rep 5: 16810). AgRP neuronal synaptic input is rapidly modulated by sensory detection of food (Mandelblat-Cerf et al., 2015, Elife 4: e07122, Betley et al., 2015, Nature 521: 180-185; Pinto et al., 2004, Science 304: 110-115): however, the impact of bodyweight and diet on activity and synaptic modulation, particularly in female mice, is understudied.

This disclosure provides an illustration of synaptic dysfunction and hyperexcitability of AgRP neurons associated with diet-induced obesity. Disturbance of intracellular Cl⁻ concentration is shown herein to have contributed to loss of postsynaptic inhibitory GABAnergic input in AgRP neurons. It was further shown that increasing expression and function of KCC2, a chloride ion (Cl⁻) extruder, and inhibiting function of NKCC1—a Cl⁻ importer, prevented body weight gain and loss of AgRP neuronal response to GABAergic inhibition induced by high fat diet (HFD).

It is to be understood that the particular aspects of the specification are described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

### Definitions

Throughout this specification, unless the context specifically indicates otherwise, the terms “comprise” and “include” and variations thereof (e.g., “comprises,” “comprising,” “includes,” and “including”) are understood to indicate the inclusion of a stated component, feature, element, or step or group of components, features, elements or steps but not the exclusion of any other component, feature, element, or step or group of components, features, elements, or steps. Any of the terms “comprising”, “consisting essentially of”, and “consisting of” may be replaced with either of the other two terms, while retaining their ordinary meanings As used herein, the singular forms “a,” “an,” and “the” include plural referents unless the context clearly indicates otherwise.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein and in the drawings, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Subject” or “patient” as used herein are used interchangeably and refer to a warm-blooded animal such as a mammal, preferably a human, which is afflicted with, or has the potential to be afflicted with DIO as described herein. The subject can be a human patient that was at risk for, or suffering from, DIO.

“Express” or “expression” as used herein refers to transcription and translation of a nucleic acid coding sequence resulting in production of the encoded polypeptide.

### Compositions

“Pharmaceutical composition” as used herein refers to a composition that includes one or more therapeutic agents, a pharmaceutically acceptable carrier, a solvent, an adjuvant, and/or a diluent, or any combination thereof. The exact nature of the carrier, solvent, adjuvant, or diluent will depend upon the desired use of the composition (e.g., route of administration), and can range from being suitable or acceptable for veterinary uses to being suitable or acceptable for human use.

“Obesity” as used herein refers to abnormal excessive fat accumulation that increases the risk of health problems. “Diet-induced obesity” is an animal model of obesity in which the animals are provided with high fat/high calorie diet to increase body weight.

“Inhibitors” as used herein refers to biologically active compounds that reduce a protein's function or expression. “Activators” as used herein refers to biologically active compounds that increase a protein's function or expression.

Reduction of Na-K-Cl co-transporter 1 (NKCC1) function can be achieved by pharmacological inhibitors or by inhibiting expression of solute carrier family 12 member 2 (Slc12a2) gene encoding NKCC1. The pharmacological inhibitors include, but are not limited to, bumetanide, azosemide, DIMAEB, or BUM66. Method to inhibit expression of Slc12a2 gene includes CRISPR-Cas9 complex with gRNA that targets Slc12a2 (NCBI gene IDs: 20496 or 6558).

Similarly, increasing K-Cl co-transporter 2 (KCC2) function can be achieved by pharmacological activators or by upregulating expression of solute carrier family 12 member 2 (Slc12a5) gene encoding KCC2. The pharmacological activators include, but are not limited to, CLP257 or its prodrug CLP290. Methods to upregulate expression of Slc12a5 gene include CRISPR activation-Cas9 complex (see, Casas-Mollano et al., 2020, CRISPR J. 3(5): 350-364) with gRNA targeting Slc12a5 (NCBI gene IDs: 57138 or 57468).

Examples of compositions appropriate for such therapeutic applications include preparations for parenteral, subcutaneous, transdermal, intradermal, intramuscular, intravenous (e.g., injectable), intrajoint, intratendon, intraligament, intrasynovial, extrasynovial, or intratracheal administration, such as sterile suspensions, emulsions, and aerosols; most beneficially pharmaceutical compositions as disclosed herein are formulated for efficient and effective delivery to AgRP-expressing neurons in the central nervous system. In some cases, pharmaceutical compositions appropriate for therapeutic applications may be in admixture with one or more pharmaceutically acceptable excipients, diluents, or carriers such as sterile water, physiological saline, glucose, or the like. For example, the compounds described herein can be administered to a subject as a pharmaceutical composition comprising a carrier solution. In some cases, pharmaceutical compositions are lyophilized. In other cases, pharmaceutical compositions as provided herein contain auxiliary substances such as wetting or emulsifying agents, pH buffering agents, gelling or viscosity enhancing additives, preservatives, flavoring agents, colors, and the like, depending upon the route of administration and the preparation desired. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

A variety of dosage schedules are contemplated by this disclosure. For example, a subject can be dosed monthly, every other week, weekly, daily, or multiple times per day. Dosage amounts and dosing frequency can vary based on the dosage form and/or route of administration, and the age, weight, sex, and/or severity of the subject's disease.

### Pharmaceutical Formulations and Methods of Treatment

In exemplary embodiments, a KCC2 activator and/or a NKCC1 inhibitor are/is provided to a subject as part of a pharmaceutical composition comprising the activator or the inhibitor, respectively, and a pharmaceutically acceptable carrier. The pharmaceutical composition can be delivered to the subject via oral administration, or via intravascular administration, such as intravenous, intramuscular, or intra-arterial administration, intraperitoneal administration, and the like. Most beneficially pharmaceutical compositions as disclosed herein are formulated for efficient and effective delivery to AgRP-expressing neurons in the central nervous system. The formulation of the pharmaceutical composition can be designed for immediate release, sustained release or delayed release.

As used herein, “treatment” refers to the clinical intervention made in response to a disease, disorder, or physiological condition of the subject or to which a subject can be susceptible. The aim of treatment includes the alleviation or prevention of symptoms, slowing or stopping the progression or worsening of a disease, disorder, or condition and/or the remission of the disease, disorder, or condition.

The terms “effective amount” or “therapeutically effective amount” refer to an amount sufficient to effect beneficial or desirable biological and/or clinical results. In other words, a “therapeutically effective” amount is an amount that will provide some alleviation, mitigation, or decrease in at least one clinical symptom in the subject.

Unless otherwise defined, all technical terms used herein have the same meaning as commonly understood by a person of ordinary skill in the art to which this disclosure belongs.

For the purposes of promoting an understanding of the principles of the disclosure, reference will now be made to embodiments and specific language will be used to describe the same. It will nevertheless be understood that no limitation of the scope of the disclosure was thereby intended, such alteration and further modifications of the disclosure as illustrated herein, being contemplated as would normally occur to one skilled in the art to which the disclosure relates.

Various exemplary embodiments of compositions and methods according to this invention are now described in the following non-limiting Examples. The Examples are offered for illustrative purposes only and are not intended to limit the scope of this invention in any way. Indeed, various modifications of the invention in addition to those shown and described herein will become apparent to those skilled in the art from the foregoing description and the following examples and fall within the scope of the appended claims.

### EXAMPLES

### Materials and Methods

### Animals

The following transgenic strains were used in the experiments disclosed herein: hrGFP-NPY (JAX Stock #006417), C57BL/6J (JAX Stock#000664), and Vgat-IRES-Cre (JAX Stock #028862). Founder mice were obtained from the JAX Repository and maintained by backcrossing with C57BI/6J. The hrGFP-NPY mice were SNP-typed to confirm that the strain was on a congenic C57Bl/6J background except for a transgene insertion site on Chr 7. Mice were maintained at 22-24 °C on a 12 h:12 h light/dark cycle (0600-1800). All mice used for breeding were fed standard lab chow (UTHSC—Teklad 7912:3.1 kcal/g metabolizable energy, 17 kcal % fat or JAX—LabDiets 5KOQ: 3.15 kcal/g metabolizable energy, 16.8 kcal % from fat). Mice were weaned at 21 days and group-housed with same sex litter mates (n=2-5 mice/pen). At 8 weeks of age, offspring were randomly assigned to either standard chow or a high-fat diet (HFD; Research Diets D12451: 4.73 kcal/g metabolizable energy, 45 kcal % from fat) and maintained on the assigned diet for 8-10 weeks. Water was available ad libitum. Mice were weighed weekly and all mice were 10-18 weeks of age for all experiments.

### Electrophysiology

Slice preparation: For all experiments, brain slices were prepared between 09:00 and 10:30 to minimize mouse-to-mouse variability and to provide AgRP neurons having the lowest activity due to overnight feeding. Mice (10-18 weeks old) used for electrophysiology experiments were deeply anesthetized using isoflurane before decapitation and rapid brain removal. Brains were then submerged in ice-cold, oxygenated (95% O₂/5% CO₂) cutting solution (in mM: 119 NaCl, 90 sucrose, 2.5 KCl, 1 MgSO₄, 2 CaCl₂, 1.25 NaH₂PO₄, 23 NaHCO₃, and 10 glucose). Coronal slices (250 μm) were cut using a vibratome (VT1000S, Leica) and incubated in oxygenated aCSF (in mM: 119 NaCl, 2.5 KCl, 1 MgSO₄, 2 CaCl₂, 1.25 NaH₂PO₄, 23 NaHCO₃, and 10 glucose) for at least 1 h prior to recording.

Slice recording: Slices were transferred to a recording chamber constantly perfused (~2 ml/min) with oxygenated aCSF. GFP-positive AgRP/NPY neurons were identified using epifluorescence and standard GFP filters on a fixed-stage SliceScope 1000 microscope (Scientifica, Uckfield, UK) equipped with a digital camera (Q-Imaging, Surry, BC, Canada). All recordings were performed using a Multiclamp 700B amplifier and Digidata 1550A, controlled using Clampex 10.7 (Molecular Devices, San Jose, CA, USA). Data were digitized at 20 kHz and filtered at 5 kHz using the built-in four-pole Bessel filter of the Multiclamp 700B.

Recording pipettes were pulled from filamented thin-wall borosilicate glass (TW150F-4, World Precision Instruments) and had a resistance of 4-7 MΩ when filled with internal solution (for intrinsic excitability (AP) recordings, in mM: 130 K-gluconate, 10 KCl, 0.3 CaCl₂, 1 MgCl₂, 1 EGTA, 3 MgATP, 0.3 NaGTP, and 10 HEPES, pH 7.35 with KOH; for synaptic recordings, in mM: 140 KCl, 0.3CaCl₂, 1 MgCl₂, 1 EGTA, 3 MgATP, 0.3 NaGTP, and 10 HEPES, pH 7.35 with KOH; for perforated patch, Gramicidin A (Sigma) was dissolved in DMSO to a concentration of 5 mg/mL and then diluted in the synaptic internal solution (140 mM KCI) to a final concentration of 20 ug/mL). The liquid junction potential (LJP) between normal aCSF and the K-gluconate solution used for intrinsic recordings was +14.7 mV and was corrected. The LJP between aCSF and the KCl intracellular solution was +4.75 mV and was not corrected.

Whole-cell current clamp recordings of resting membrane potential and spontaneous firing were recorded in the presence of DNQX (10 μM; Tocris) and picrotoxin (100 μM; Tocris). For experiments testing inhibition of AgRP neurons by leptin, mice were fasted overnight to promote intrinsic excitability and 100 nM leptin (National Hormone and Peptide Program) was bath applied. Whole-cell voltage-clamp recordings for mEPSC and mIPSC were conducted in the presence of TTX (1 μM; Tocris) and picrotoxin (100 μM; Tocris) for mEPSCs and 6,7-Dinitroquinoxaline-2,3-dione (10 μM DNQX; Tocris) for mIPSCs. Synaptic frequency, inter-event interval, amplitude, and τ-decay for mEPSC and mIPSC recordings were measured.

Perforated patch voltage-clamp recordings for EGABA were conducted in the presence of DNQX. Giga-Ohm seals were quickly established with target cells, then EGABA measurements were made after pipette resistance dropped to values between 40-80 MΩ, typically 15-45 min. Once resistance was stable, cells were voltage clamped at −70 mV, and +15 mV voltage steps were applied, ranging from −80 mV to 10 mV during which GABA (100 μM) was focally applied via pressure ejection using a Picospritzer III (Parker Hannifin, Hollis, NH USA) or PDES Pneumatic drug Ejection (npi electronic GmbH, Tamm, Germany). The I/V curve for each cell was calculated based on the current (pA) immediately prior to and following the GABA-puff.

Cell-attached recordings were conducted in voltage-clamp mode in the presence of picrotoxin (100 μM) for glutamate experiments and DNQX (10 μM) for GABA experiments. Normal aCSF was used as the intracellular solution. For both experiments, a 10-second puff of either glutamate (100 μM) or GABA (100 μM) were applied following establishment of a loose-patch (15-75 MΩ). Sections were incubated (>45 min) and bath perfused with either bumetanide (10 μM) or DMSO (10 μM).

### Intra-Arcuate Viral Injections

For intra-arcuate adeno-associated virus (AAV) delivery, mice were anesthetized to effect using 1-2.5% isoflurane and placed in a stereotaxtic frame (Kopf Instruments). F1 offspring from a cross between Vgat^{Cre} and hrGFP-NPY mice were used for all optogenetic experiments. The skull was exposed and a burr hole drilled above the target region (vDMH). One hundred nanoliters (nL) of AAV9-EFla-double floxed—hChR2(H134R)-mCherry (Addgene) was injected at a rate of 50 nL/min using a UMP3 syringe pump (World Precision Instruments) into the vDMH (coordinates: AP=−1.5 mm, DV=−5.2 mm, ML=±0.3 mm from bregma). The injection needle remained in place for 5 minutes before retraction, after which the incision was closed using non-absorbable 6.0 silk and the mice were allowed to recover. All surgeries were performed unilaterally and mice were used for slice electrophysiology experiments 3-4 weeks post-injection.

### Data Analysis and Statistics

Post-synaptic current frequencies, amplitudes, inter-event intervals and decay time constants were measured using Clampfit 10.7 (Molecular Devices) and Axograph (AxoGraph, Inc). I/V curves from perforated patch recordings were calculated in Clampfit 10.7 (Molecular Devices). Statistical outliers were identified using the ROUT method (Q=1% cutoff threshold) as implemented in GraphPad Prism 9. Group differences were analyzed with two-way ANOVA followed by Tukey's multiple comparisons post hoc test using Prism 8 and 9 (GraphPad). When required, three-way ANOVA was performed using the aov function in R (v3.6.3) using the R package r/emmeans (available online https://cran.r-project.org/web/packages/emmeans/index.html) for post hoc Tukey's multiple comparison testing with Bonferroni's correction. For repeated measures analysis, group differences were analyzed by two-way repeated measures-ANOVA using SPSS (IBM) or R. Data visualization was performed using Prism or r/ggplot2. For all statistical tests, a value of p<0.05 was considered significant. Data are presented as the mean±SEM; violin plots are presented as median +quartile.

### Example 1: Age and Sex Influenced AgRP Neuron's Intrinsic Excitability In Diet-Induced Obesity

Inclusion of females in mechanistic studies of hypothalamic function remains low and very little is known about how diet or metabolic state influences AgRP neuronal function in females, despite involvement in a variety of female-specific domains including fertility (Egan et al., 2017, J Neurosci 37: 3875-3886; Padilla et al., 2017, Proc Natl Acad Sci USA 114: 2413-2418), nest-building (Li et al., 2019, J Neurosci 39:456-471; Phillips and Palmiter, 2008, Endocrinology 149: 544-550) and lactation (Cavalcanti-de-Albuquerque et al., 2019, Nat Commun 10: 311). Further, metabolism and substrate preference are sexually dimorphic; females tend to rely on lipolysis and fatty acid metabolism while males preferentially use carbohydrates (Yao et al., 2010, Biochim Biophys Acta 1800: 1121-1126; Hedrington and Davis, 2015, Front Endocrinol 6: 61; Klosinski et al., 2015, EBioMedicine 2, 1888-1904). Distribution of energy stores and patterns of energy utilization during weight loss are also sexually dimorphic (Pietrobelli et al., 2002, Int J Obes Relat Metab Disord 26: 1339-1348; Palmer and Clegg, 2015, Mol Cell Endocrinol 402: 113-119), and when considered with the recent finding that AgRP neurons regulate substrate utilization in metabolism and lipogenesis (Cavalcanti-de-Albuquerque et al., 2019, Nat Commun 10: 311), sex-dependent differences in AgRP neuronal function may reflect one mechanism by which females regulate metabolic flexibility, thus emphasizing the importance of understanding how diet and obesity influence the intrinsic and synaptic excitability of AgRP neurons in female mice.

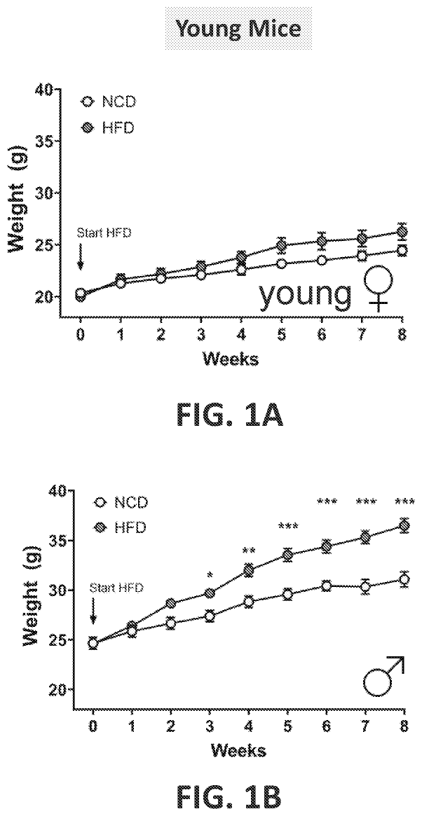

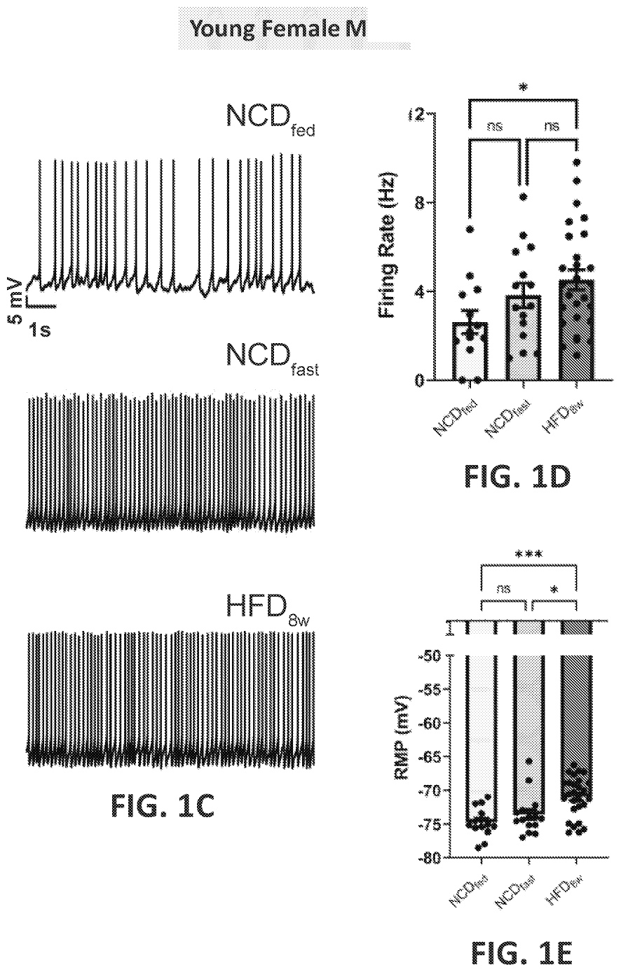

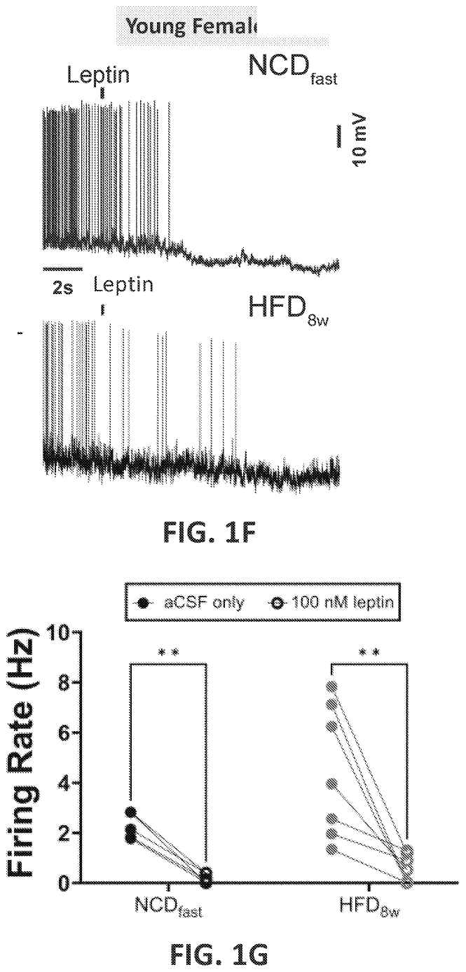

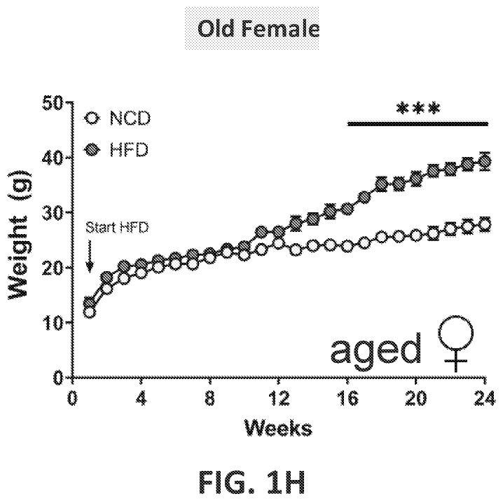

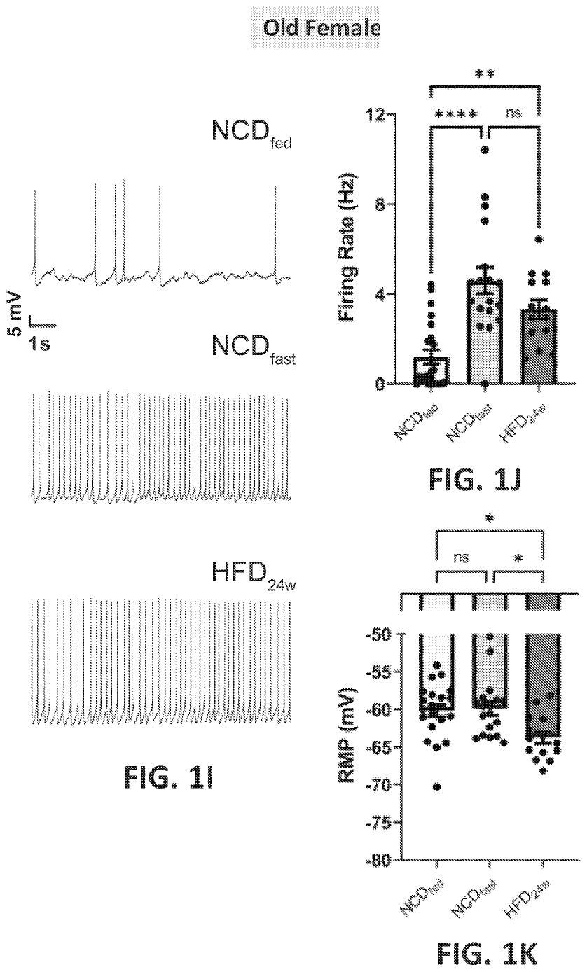

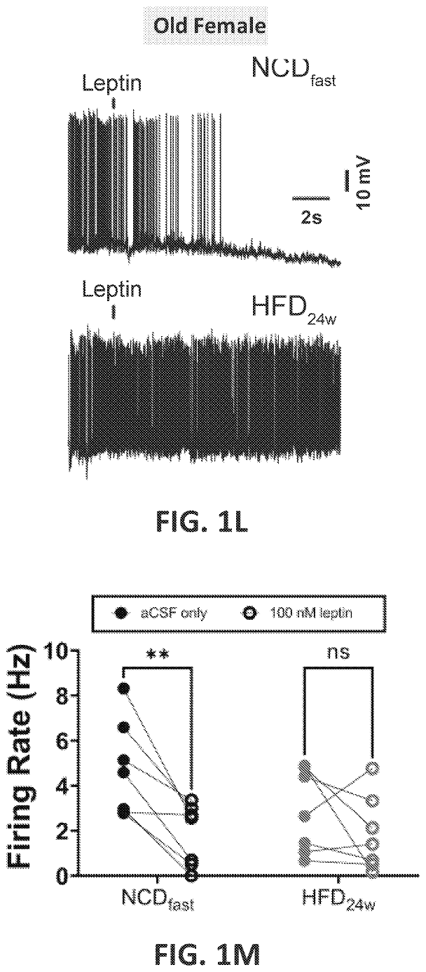

To investigate the impact of gender and HFD on bodyweight, AgRP neuronal excitability, and leptin sensitivity, male and female 8-week-old C57BI/6J mice were fed a low-fat control normal chow diet (NCD) or HFD for 8 weeks. Regardless of diet, male mice weigh more than females at every age, with gender accounting for the greatest proportion of variation in bodyweight (45.7%). Consistent with prior reports (Baver et al., 2014, J Neurosci 34: 5486-5496; Salinero et al., 2018, Int J Obes 42: 1088-1091), HFD_{fed} male mice exhibited diet-induced weight gain beginning at ~2 weeks (FIG. 1A), while HFD_{fed} female mice exhibited minimal weight gain over 8 weeks with no difference between NCD- and HFD-fed groups at any time (Salinero et al., 2018, Int J Obes 42: 1088-1091; Hong et al., 2009, Nutr J 8: 11; Hwang et al., 2010, Obesity 18: 463-469; Atamni et al., 2016, BMC Genet 17: 10; Dorfman et al., 2017, Nat Commun 8: 14556) (FIG. 1B).

In HFD_{fed} male mice, AgRP neurons were known to become hyperexcitable and refractive to modulation by physiological cues of hunger and satiety (Baver et al, 2014, J Neurosci 34: 5486-5496). To determine whether resistance to diet-induced obesity (DIO) in female mice accompanied resistance to diet-induced remodeling of AgRP neuronal excitability, intrinsic properties of AgRP neurons from NCD in fast and fed state and HFD_{8w} in fed state in female mice were measured (FIG. 1C). HFD_{8w} in fast state was not measured because previous report showed that in mice with HFD, fasting did not alter the excitability of AgRP neurons from fed state (Baver et al., 2014, J Neurosci 34: 5486-5496). The baseline activity of AgRP neurons in NCD_{fed} mice was found to be dimorphic, with neurons from lean female mice exhibiting a significantly higher baseline firing rate compared to neurons from males. Despite this, in female mice, overnight fasting in NCD group and feeding in HFD_{8w} group slightly increased AgRP neuronal firing rate (FIG. 1D). An additional measure of intrinsic excitability, the resting membrane potential (RMP) was found to be depolarized in HFD_{8w} but not NCD_{fast} females (FIG. 1E). Leptin sensitivity in AgRP neurons was assessed as set forth in Baver et al. (2014, J Neurosci 34: 5486-5496), wherein 100 nM leptin was found to inhibit AgRP neuronal activity in brain slices from NCD_{fast} and HFD_{8w} females (FIG. 1F and FIG. 1G). Taken together, the data revealed robust sexual dimorphism in both baseline AgRP neuronal function and response to HFD, with female mice exhibiting resistance to DIO and increased neuronal activity independent of bodyweight.

To determine when female mice began to exhibit DIO, a separate cohort of female mice was fed HFD for 24 weeks. Consistent with a recent report that middle-aged female mice (30-40 weeks old) develop obesity when fed a 60kcal % HFD (Salinero et al., 2018, Int J Obes 42: 1088-1091), female mice exhibited significantly increased body weight after 16 weeks of HFD feeding (FIG. 1H). It was then determined whether excitability of AgRP neurons after HFD_{24w} was altered in female mice. AgRP neuronal firing from older (8 m old), NCD_{fed} female mice was found to be significantly lower than in young (4 m old) female mice (FIG. 1I vs FIG. 1C, and FIG. 1J vs FIG. 1D) and was more similar to young (4 m old) male mice. It was reported previously (Baver et al., 2014, J Neurosci 34: 5486-5496; Wei et al., 2015, Sci Rep 5: 16810) that in male mice, both fasting and HFD increased the firing rate of AgRP neurons, which coincided with the results found with firing rate in older female mice (FIG. 1J). However, in these older females, fasting did not result in depolarization of the RMP of AgRP neurons and HFD_{24w} feeding induced a hyperpolarizing shift in the RMP (FIG. 1K), suggesting that both age and gender impacted the function of AgRP neurons both at baseline and in response to HFD. As in young male and female mice, application of 100 nM leptin resulted in inhibition of AgRP neuronal firing (FIG. 1L and FIG. 1M) in brain slices from older NCD female mice. Unlike in young female mice on HFD for 8 weeks, AgRP neurons from older HFD_{24w} female mice were no longer inhibited by leptin, indicating that hyperexcitability of AgRP neurons in older, DIO female mice was accompanied by development of central leptin resistance (FIG. 1L and FIG. 1M) similar to young male mice (Baver et al., 2014, J Neurosci 34: 5486-5496).

Taken together, the data showed that HFD promoted overall hyperexcitability in male mice regardless of age, while in female mice this effect was only prominent in old age.

### Example 2: Long-Term HFD had a Minimal Impact on Excitatory Input but Resulted in Increased Inhibitory Input to AgRP Neurons

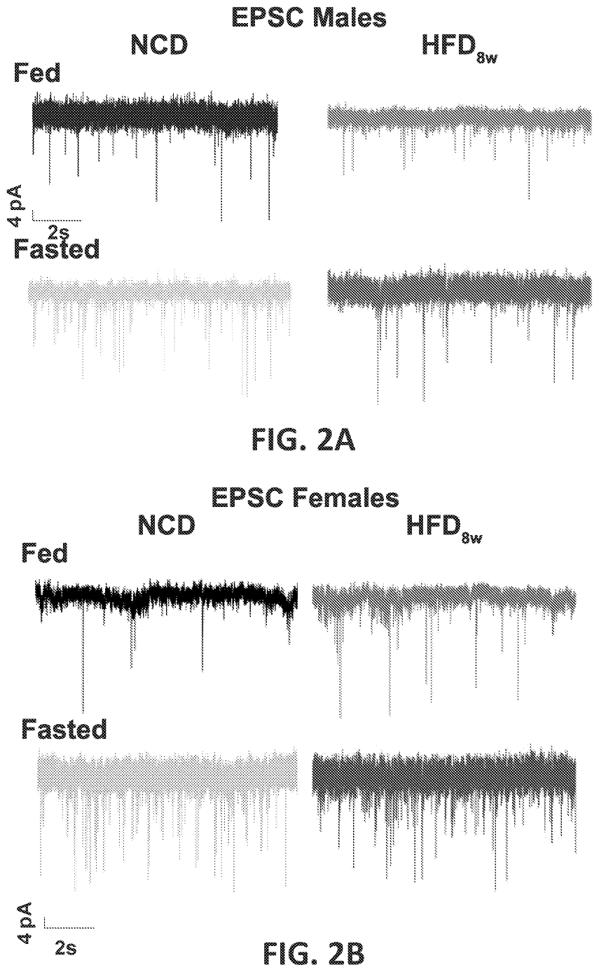

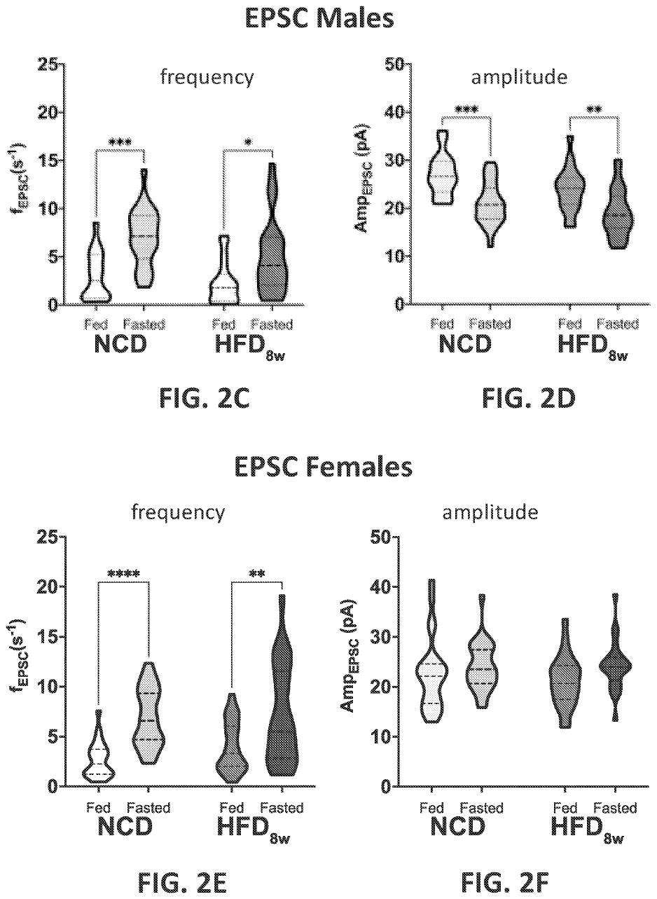

Whether long-term HFD had a more drastic effect on synaptic inputs of AgRP neurons was determined. Because AgRP neurons were known to be activated by hunger, experiments were carried out in a fed state as a control (baseline), and in a fasted state to trigger a response in AgRP neurons. Both male and female mice were fed NCD (control) or HFD for 8 weeks (HFD_{8w}), and the response to fasting was measured. Consistent with previous reports (Liu et al., 2012, Neuron 73: 511-522; Yang et al., 2011, Cell 146: 992-1003) in control diet (NCD), f_{mEPSC} increased in response to fasting in all animals (FIG. 2A, FIG. 2B, FIG. 2C, and FIG. 2E). HFD_{8w} feeding did not significantly alter the baseline f_{mEPSC} in neurons from fed male and female mice (FIG. 2A, FIG. 2B, FIG. 2C, and FIG. 2E), nor did it affect the response to fasting as shown by an increase in f_{mEPSC} with fasting in HFD for both males and females (FIG. 2A, FIG. 2B, FIG. 2C, and FIG. 2E). In male mice, fasting was associated with a significant decrease in the Amp_{mEPSC} regardless of diet (FIG. 2A and FIG. 2D), an effect not seen in AgRP neurons from female mice (FIG. 2B and FIG. 2E). HFD did not change the response to fasting in both males and females (FIG. 2D and FIG. 2F). Taken together, these results suggested that neither HFD nor obesity impacted excitatory glutamatergic presynaptic input (reflected by f_{mEPSC}) to AgRP neurons or metabolic state-dependent synaptic plasticity, and only very slightly alters the postsynaptic response (reflected by Amp_{mEPSC}) to excitatory input.

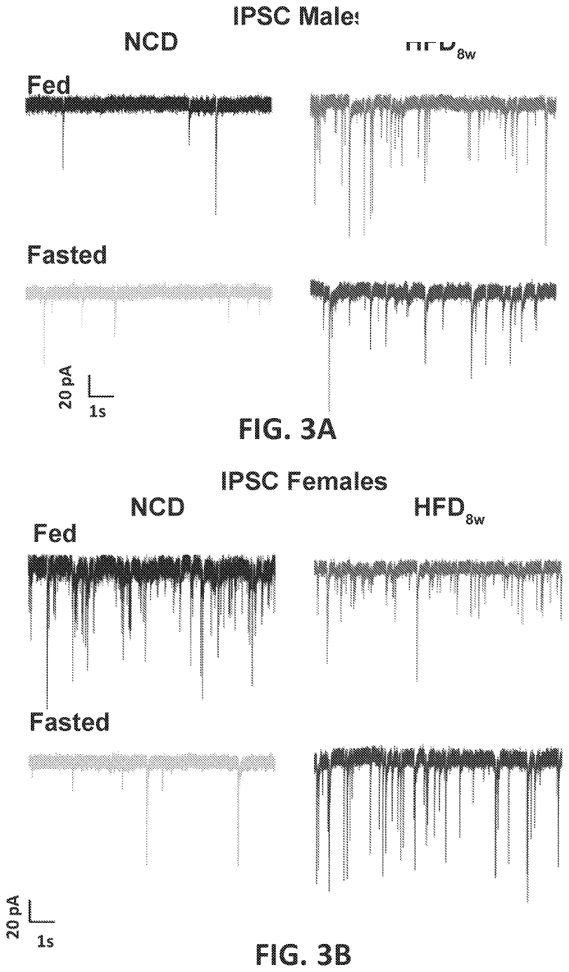

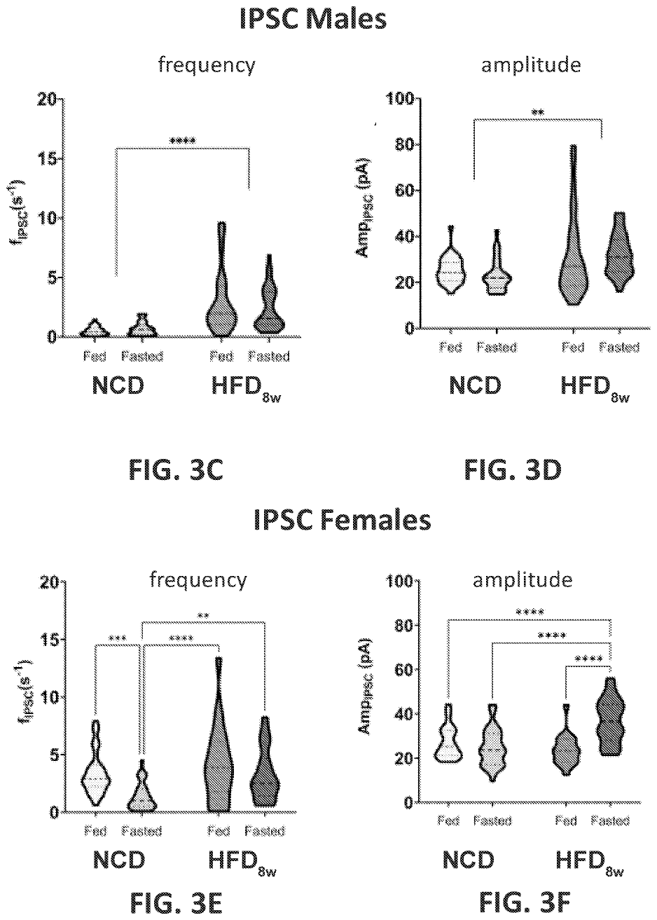

Without an effect of HFD_{8w} on excitatory glutamatergic input to AgRP neurons, whether diet-induced hyperexcitability of AgRP neurons was due to disruption of excitation/inhibition (E/I) balance was investigated. AgRP neurons have been known to be rapidly inhibited by the sensory detection of food (Mandelblat-Cerf et al., 2015, Elife 4: e07122; Betley et al., 2015, Nature 521: 180-185; Chen et al., 2015. Cell 160: 829-841), a response that was blunted in HFD_{fed} mice (Beutler et al., 2020, Elife 9: e55909; Mazzone et al., 2020, Nat Neurosci. 23(10): 1253-1266), suggesting that 1) inhibitory input is a key factor in the physiological regulation of AgRP neurons and 2) HFD and/or DIO alters this input. In AgRP neurons from male mice, under control diet, f_{mIPSC} was low and unresponsive to fasting (FIG. 3A, FIG. 3C, and FIG. 3D). However, in female, f_{mIPSC} was high in fed state compared to male and decreased in response to fasting under control diet (FIG. 3A vs FIG. 3B). In neurons from male mice, there was a significant effect of diet on f_{mIPSC} in AgRP neurons, as HFD_{8w} consumption increased the f_{mIPSC} in AgRP neurons in both fed and fasted state compared to control diet (FIG. 3A and FIG. 3C). Although HFD did not alter the f_{IPSC} in neurons from fed female mice, the response to fasting in female mice was altered by HFD, as f_{mIPSC} was not significantly different between HFD_{fed} and HFD_{fast} neurons (FIG. 3E). With regards to amplitude, fasting had no effect under control NCD in all animals (FIG. 3D and FIG. 3F). Interestingly, when HFD was introduced, fasting increased the amplitude from baseline/fed state in female (FIG. 3F); while in males, fasting still had no effect but the amplitude in baseline and fasting state were higher compared to those under NCD (FIG. 3D).

Taken together, this data suggested that long-term consumption of HFD leading to obesity increased inhibitory input in males; while in females, HFD blunted the response to fasting in the pre-synaptic inhibitory input (f_{mEPSC} in FIG. 3E) and increased response to fasting in the post-synaptic inhibitory input (Amp_{mEPSC} in FIG. 3F).

### Example 3: Long-Term HFD Blunted AgRP Neuronal Response to Postsynaptic Inhibitory GABAnergic Input

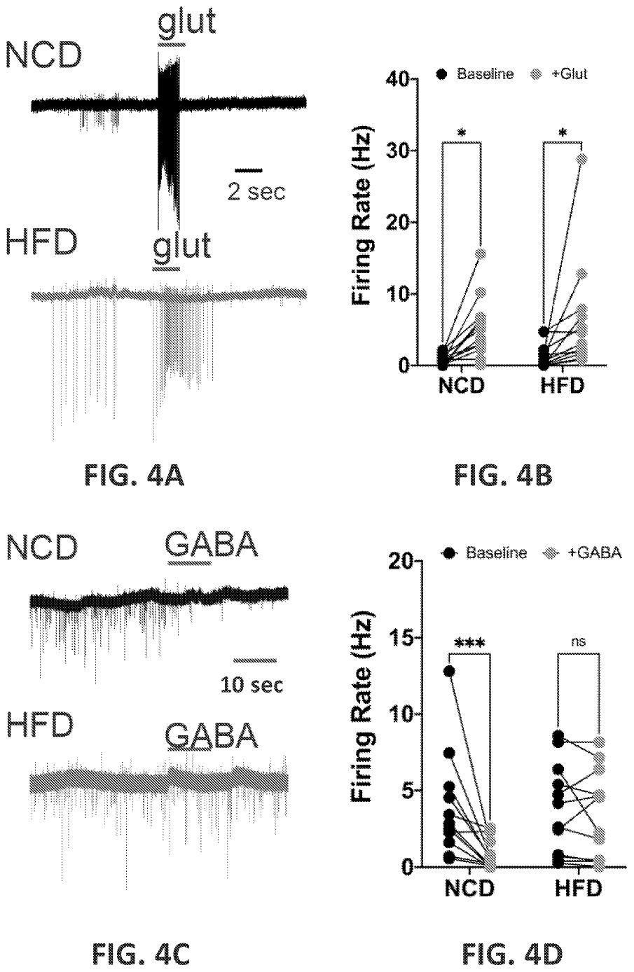

These data suggested that AgRP neurons received both excitatory and inhibitory synaptic input and unexpectedly, it was found in Example 2 that HFD had a minimal effect on post-synaptic excitatory input but enhanced the post-synaptic inhibitory input in response to fasting, despite the overall increased intrinsic excitability in AgRP neurons with long-term HFD as shown in Example 1. In other words, the postsynaptic response to these excitatory and inhibitory inputs was decoupled from intrinsic excitability, and in a sexually dimorphic manner. To test whether glutamate can stimulate firing of AgRP neurons, spontaneous firing rates of AgRP neurons from NCD_{fed} and HFD_{fed} mice were recorded in response to a brief (2 s) puff of 10 μM of 10 μM glutamate using cell-attached configuration to maintain physiological ionic gradients. There was no effect of diet on direct glutamate stimulation, and neurons from NCD_{fed} and HFD_{fed} mice were excited by glutamate (FIG. 4A and FIG. 4B). However, 10 s puff application of 100 μM GABA decreased the firing rate of neurons from NCD_{fast} mice (FIG. 4C and FIG. 4D), but had no effect on the firing rate of neurons from HFD_{fed} mice. Surprisingly, in 25% of HFD neurons (3 of 12), GABA application resulted in an increase in the firing rate (FIG. 4D), indicating that the intracellular Cl-gradient is disrupted by HFD in AgRP neurons.

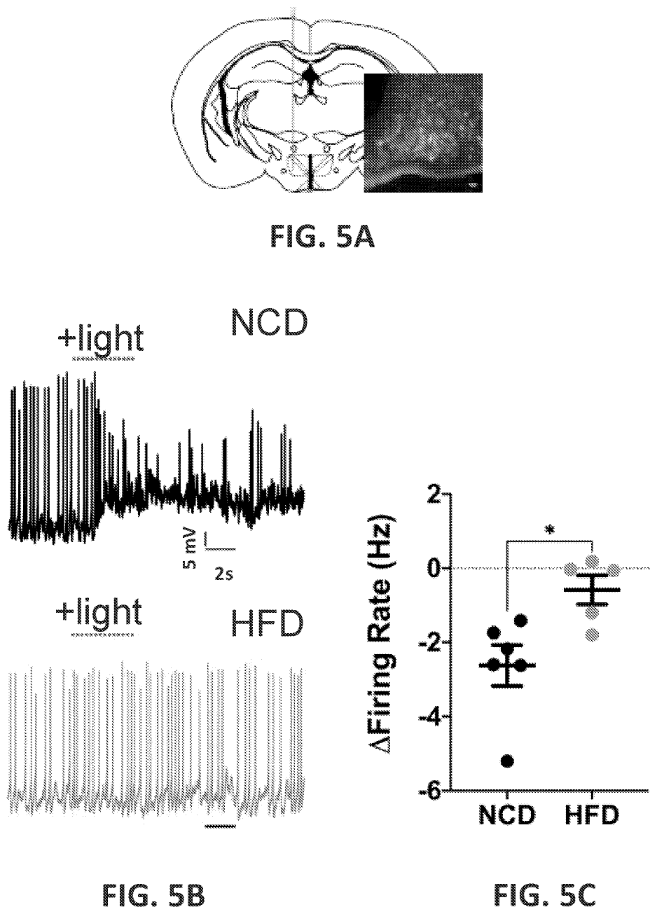

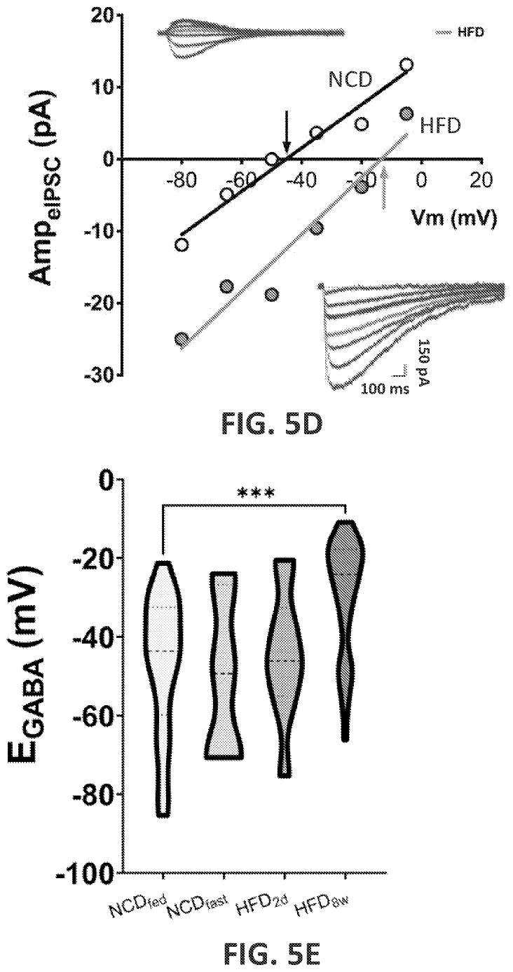

To confirm that AgRP neurons from HFD mice were not inhibited by GABA in a physiological context, light-activated cation channel rhodopsin 2 (ChR2) was expressed in GABAergic Lepr⁺ neurons in the ventral dorsomedial hypothalamus (vDMH), a population that directly inhibits AgRP neurons and suppresses food intake (Garfield et al., 2016, Nat Neurosci 19:1628-1635) (FIG. 5A). Using whole-cell patch clamp to monitor spontaneous firing of AgRP neurons during photostimulation of ChR2-mCherry⁺ terminals from vDMH^{Lepr} neurons in the ARH (to specifically activate the GABAergic inputs from the vDMH), brain slices from NCD_{fast} mice rapidly inhibited firing in the postsynaptic AgRP neurons during photoactivation of vDMH terminals. However, in HFD_{fed} mice, photostimulation had no significant effect on the firing rate of AgRP neurons (FIG. 5B and FIG. 5C).

### Example 4: Dysfunction of KCC2 and Dissipation of Intracellular Cl⁻ Ionic Gradient Underlie Loss of Postsynaptic GABAnergic Inhibition in AgRP Neurons

In adult neurons, GABA functions as an inhibitory neurotransmitter because intracellular Cl⁻ concentrations are maintained at a very low level due to the activity of the K⁺-Cl⁻ cotransporter KCC2 (Slc12a5), resulting in a hyperpolarized reversal potential for the GABA-evoked Cl⁻ current (E_{GABA}). Therefore, it was determined whether HFD caused dysfunction in KCC2 activity, leading to dissipation of the neuronal Cl⁻ gradient and a loss of GABAergic inhibition. KCC2 is an electroneutral transporter, precluding direct electrophysiological assessment of its function; thus, KCC2 function and the Cl⁻ gradient in AgRP neurons was indirectly assessed by measuring EGABA using gramicidin perforated patch. EGABA in AgRP neurons from NCD_{fed} mice was −47.09±3.6 mV with no significant shift in EGABA in AgRP neurons from either NCD_{fast} or short-term HFD_{2d} (FIG. 5E) However, consistent with the observation that GABA no longer inhibited AgRP neurons from long-term HFD_{8w} mice in Example 3 above, there was a significant depolarizing shift in the GABA reversal potential (FIG. 5D and FIG. 5E).

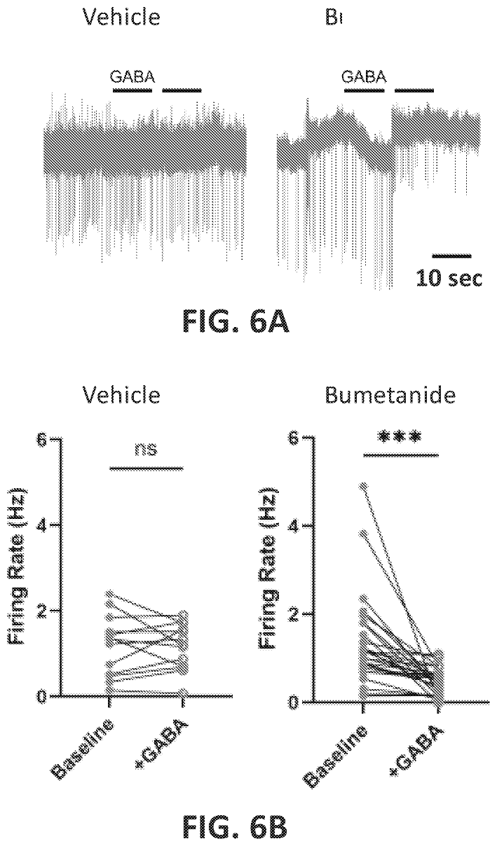

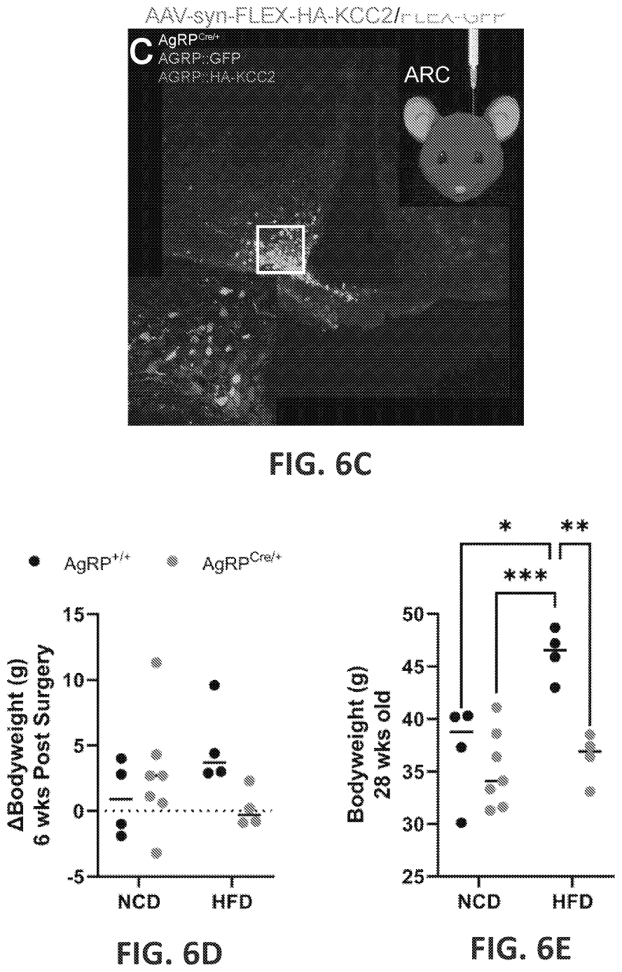

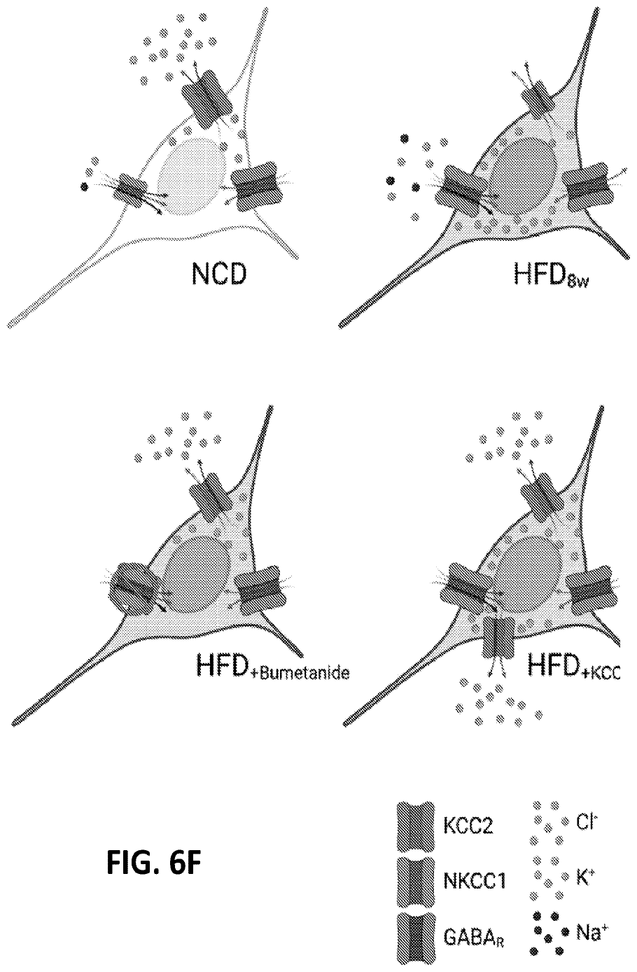

Based on these results, dysfunction of the neuronal K⁺/Cl⁻ cotransporter KCC2 and downstream impact on neuronal Cl⁻ homeostasis was suggested to underlie many of the observed effects of HFD on AgRP neuronal excitability. In the brain, intracellular Cl⁻ concentration is largely determined by the Cl⁻ importer NKCC1 (SLC12A2) and the Cl⁻ exporter KCC2 (SLC12A5). KCC2 is the primary Cl⁻ extruder in mature neurons, including AgRP neurons and is responsible for keeping intraneuronal Cl⁻ low, forming the biophysical basis for the inhibitory action of GABA_{A}-R. The importance of this function is best exemplified by the switch of GABA from excitatory to inhibitory subsequent to upregulation of KCC2 during development, however, it is increasingly apparent that KCC2 dysfunction is a contributing factor in several adult-onset disorders, including epilepsy (Moore et al., 2018, Proc Natl Acad Sci USA 115:10166-10171; Moore et al., 2017, Trends Neurosci 40: 555-571), ischemic stroke (Jaenisch et al., 2010, Stroke 41: e151-159), Huntington's disease (Dargaei et al., 2018, Proc Natl Acad Sci USA 115: E1618-E1626), and peripheral nerve injury (Boulenguez. et al., 2010, Nat Med 16: 302-307; Chen et al., 2018, Cell 174: 521-535 e513). NKCC1 expression is increased in these conditions (and few effective activators of KCC2 exist), and pharmacological inhibition of NKCC1 with bumetanide has successfully confirmed pathological dysregulation of neuronal Cl⁻ levels and restored GABA-mediated inhibition (Boulenguez et al., 2010, Nat Med 16: 302-307; Savardi et al., 2021, Trends Pharmacol Sci 42(12: 1009-1034); Ben-Ari and Cherubini, 2022, Cells 11(3): 396). Therefore, bumetanide treatment of brain slices from DIO mice were expected from the results disclosed herein to restore the inhibitory action of GABA by blocking Cl⁻ import via NKCC1 (FIG. 6F). To establish this expectation, ARH slices were incubated in either aCSF+DMSO or aCSF+bumetanide for >45 min to block NKCC1 activity (Dargaei et al., 2018, Proc Natl Acad Sci USA 115: E 1618-E1626; Khirug et al., 2010, J Neurosci 30: 12028-12035). AgRP neuronal excitability was then measured using cell-attached patch recording and 100 μM GABA applied locally using puff application. As before, AgRP neurons from HFD_{8w} mice in brain slices pretreated with aCSF+DMSO were not inhibited by focal application of GABA (FIG. 6A). However, following incubation with bumetanide, AgRP neurons from HFD_{8w} mice were inhibited by GABA (FIG. 6B), suggesting that dyshomeostasis of intraneuronal Cl⁻ in AgRP neurons was a causal factor in diet-induced hyperexcitability of AgRP neurons.

To examine the impact of KCC2 expression on HFD-induced weight gain, AAV-PHP. B-FLEX-KCC2 (Chen et al., 2018, Cell 174: 521-535 e513) (which directed Cre-dependent KCC2 expression in recipient neurons) was injected into the arcuate nucleus (ARC) of AgRP^{+/+} and AgRP^{Cre/+} mice after 2-6 weeks of HFD feeding (FIG. 6C). In contrast to AgRP⁺⁺, HFD fed AgRP^{Cre/+} mice began to show decreased weight gain 6 weeks following surgery (FIG. 6D). By 28 weeks of age (>10 weeks post-surgery, HFD_{20w} feeding) HFD increased bodyweight in AgRP^{+/+} mice, but not in AgRP^{Cre/+} mice (FIG. 6E).

### Example 5: NKKC1 Inhibition in Vivo Reduces Weight Gain Induced by HFD

To examine the impact of KCC2 expression on HFD-induced weight gain, AAV-shRNA with AgRP specific promoter construct targeting against NKCC1 or control AAV-scrambled-shRNA vector is injected into the ARC of AgRP mice after 2-6 weeks of HFD feeding. In contrast to the control group, HFD fed shRNA-NKCC1 mice begin to show decreased weight gain 6 weeks following surgery. By 28 weeks of age (>10 weeks post-surgery, HFD_{20w} feeding) HFD increases bodyweight in control mice, but not in shRNA-NKCC1 mice.

Alternatively, mice are administered control drug or bumetanide 100 mg/kg weight orally for up to 12 weeks after 2-6 weeks of HFD. Bodyweight is monitored for 12 weeks. By 20 weeks of age (>10 weeks post-surgery, HFD_{12w} feeding) HFD increases bodyweight in vehicle orally administered mice, but not in bumetanide group.

Taken together these results established that dysregulation of intracellular Cl⁻ concentration in AgRP neurons led to loss of postsynaptic GABAnergic inhibition in diet-induced obesity, and that manipulation of co-transporters to restore the Cl⁻ concentration is crucial to rectify the excitability of AgRP neurons and suppress weight gain induced by HFD.

All publications, patents, and patent applications mentioned in this specification areherein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, andthe appended claims. The mere fact that certain measures or features are recited in mutually different dependent claims does not indicate that the combination of these measures or features cannot be used. Any reference signs in the claims should not be construed as limiting the scope.

### REFERENCES

Berk, K. A. et al. Group cognitive behavioural therapy and weight regain after diet in type 2 diabetes: results from the randomised controlled POWER trial. Diabetologia 61, 790-799, doi: 10.1007/s00125-017-4531-9 (2018).
Fothergill, E. et al. Persistent metabolic adaptation 6 years after “The Biggest Loser” competition. Obesity (Silver Spring) 24, 1612-1619, doi:10.1002/oby.21538 (2016).
Greenway, F. L. Physiological adaptations to weight loss and factors favouring weight regain. Int J Obes (Lond) 39, 1188-1196, doi:10.1038/ijo.2015.59 (2015).
Hall, K. D. & Guo, J. Obesity Energetics: Body Weight Regulation and the Effects of Diet Composition. Gastroenterology 152, 1718-1727 e1713, doi:10.1053/j.gastro.2017.01.052 (2017).
Sumithran, P. & Proietto, J. The defence of body weight: a physiological basis for weight regain after weight loss. Clin Sci (Lond) 124, 231-241, doi:10.1042/CS20120223 (2013).
Morton, G. J. & Schwartz, M. W. The NPY/AgRP neuron and energy homeostasis. Int J Obes Relat Metab Disord 25 Suppl 5, S56-62, doi:10.1038/sj.ijo.0801915 (2001).
KÖnner, A. C. et al. Insulin action in AgRP-expressing neurons is required for suppression of hepatic glucose production. Cell Metab 5, 438-449, doi:10.1016/j.cmet.2007.05.004 (2007).
Andrews, Z. B. et al. UCP2 mediates ghrelin's action on NPY/AgRP neurons by lowering free radicals. Nature 454, 846-851, doi:10.1038/nature07181 (2008).
Varela, L. & Horvath, T. L. Leptin and insulin pathways in POMC and AgRP neurons that modulate energy balance and glucose homeostasis. EMBO Rep 13, 1079-1086, doi:10.1038/embor.2012.174 (2012).
Krashes, M. J. et al. An excitatory paraventricular nucleus to AgRP neuron circuit that drives hunger. Nature 507, 238-242, doi:10.1038/nature12956 (2014).
Garfield, A. S. et al. Dynamic GABAergic afferent modulation of AgRP neurons. Nat Neurosci 19, 1628-1635, doi: 10.1038/nn.4392 (2016).
van den Top, M., Lee, K., Whyment, A. D., Blanks, A. M. & Spanswick, D. Orexigen-sensitive NPY/AgRP pacemaker neurons in the hypothalamic arcuate nucleus. Nat Neurosci 7, 493-494, doi:10.1038/nn1226 (2004).
Takahashi, K. A. & Cone, R. D. Fasting induces a large, leptin-dependent increase in the intrinsic action potential frequency of orexigenic arcuate nucleus neuropeptide Y/Agouti-related protein neurons. Endocrinology 146, 1043-1047, doi:10.1210/en.2004-1397 (2005).
Baver, S. B. et al. Leptin modulates the intrinsic excitability of AgRP/NPY neurons in the arcuate nucleus of the hypothalamus. J Neurosci 34, 5486-5496, doi:10.1523/JNEUROSCI.4861-12.2014 (2014).
Mandelblat-Cerf, Y. et al. Arcuate hypothalamic AgRP and putative POMC neurons show opposite changes in spiking across multiple timescales. Elife 4, doi:10.7554/eLife.07122 (2015).
Aponte, Y., Atasoy, D. & Sternson, S. M. AGRP neurons are sufficient to orchestrate feeding behavior rapidly and without training. Nat Neurosci 14, 351-355, doi:10.1038/nn.2739 (2011).
Krashes, M. J. et al. Rapid, reversible activation of AgRP neurons drives feeding behavior in mice. J Clin Invest 121, 1424-1428, doi:10.1172/JCI46229 (2011).
Chen, Y., Lin, Y. C., Zimmerman, C. A., Essner, R. A. & Knight, Z. A. Hunger neurons drive feeding through a sustained, positive reinforcement signal. Elife 5, doi:10.7554/eLife. 18640 (2016).
Luquet, S., Perez, F. A., Hnasko, T. S. & Palmiter, R. D. NPY/AgRP neurons are essential for feeding in adult mice but can be ablated in neonates. Science 310, 683-685, doi:10.1126/science.1115524 (2005).
Beutler, L. R. et al. Obesity causes selective and long-lasting desensitization of AgRP neurons to dietary fat. Elife 9, doi:10.7554/eLife.55909 (2020).
Mazzone, C. M. et al. High-fat food biases hypothalamic and mesolimbic expression of consummatory drives. Nat Neurosci, doi:10.1038/s41593-020-0684-9 (2020).
Wei, W. et al. Diet composition, not calorie intake, rapidly alters intrinsic excitability of hypothalamic AgRP/NPY neurons in mice. Sci Rep 5, 16810, doi:10.1038/srep16810 (2015).
Betley, J. N. et al. Neurons for hunger and thirst transmit a negative-valence teaching signal. Nature 521, 180-185, doi:10.1038/nature14416 (2015).
Chen, Y., Lin, Y. C., Kuo, T. W. & Knight, Z. A. Sensory detection of food rapidly modulates arcuate feeding circuits. Cell 160, 829-841, doi:10.1016/j. cell.2015.01.033 (2015). Pinto, S. et al. Rapid rewiring of arcuate nucleus feeding circuits by leptin. Science 304, 110-115, doi:10.1126/science.1089459 (2004).
Egan, O. K., Inglis, M. A. & Anderson, G. M. Leptin Signaling in AgRP Neurons Modulates Puberty Onset and Adult Fertility in Mice. J Neurosci 37, 3875-3886, doi:10.1523/JNEUROSCI.3138-16.2017 (2017).
Padilla, S. L. Q., J. et al. AgRP to Kiss1 neuron signaling links nutritional state and fertility. Proc Natl Acad Sci U S A 114, 2413-2418, doi:10.1073/pnas.1621065114(2017).
Li, X. Y. et al. AGRP Neurons Project to the Medial Preoptic Area and Modulate Maternal Nest-Building. J Neurosci 39, 456-471, doi:10.1523/JNEUROSCI.0958-18.2018 (2019).
Phillips, C. T. & Palmiter, R. D. Role of agouti-related protein-expressing neurons in lactation. Endocrinology 149, 544-550, doi:10.1210/en.2007-1153 (2008).
Yao, J., Hamilton, R. T., Cadenas, E. & Brinton, R. D. Decline in mitochondrial bioenergetics and shift to ketogenic profile in brain during reproductive senescence. Biochim Biophys Acta 1800, 1121-1126, doi:10.1016/j.bbagen.2010.06.002 (2010).
Hedrington, M. S. & Davis, S. N. Sexual Dimorphism in Glucose and Lipid Metabolism during Fasting, Hypoglycemia, and Exercise. Front Endocrinol (Lausanne) 6, 61, doi:10.3389/fendo.2015.00061 (2015).
Klosinski, L. P. et al. White Matter Lipids as a Ketogenic Fuel Supply in Aging Female Brain: Implications for Alzheimer's Disease. EBioMedicine 2, 1888-1904, doi: 10.1016/j.ebiom.2015.11.002 (2015).
Pietrobelli, A. et al. Sexual dimorphism in the energy content of weight change. Int J Obes Relat Metab Disord 26, 1339-1348, doi:10.1038/sj.ijo.0802065 (2002).
Palmer, B. F. & Clegg, D. J. The sexual dimorphism of obesity. Mol Cell Endocrinol 402, 113-119, doi: 10.1016/j. mce.2014.11.029 (2015).
Cavalcanti-de-Albuquerque, J. P., Bober, J., Zimmer, M. R. & Dietrich, M. O. Regulation of substrate utilization and adiposity by Agrp neurons. Nat Commun 10, 311, doi:10.1038/s41467-018-08239-x (2019).
Salinero, A. E., Anderson, B. M. & Zuloaga, K. L. Sex differences in the metabolic effects of diet-induced obesity vary by age of onset. Int J Obes (Lond) 42, 1088-1091, doi:10.1038/s41366-018-0023-3 (2018).
Hong, J., Stubbins, R. E., Smith, R. R., Harvey, A. E. & Néñez, N. P. Differential susceptibility to obesity between male, female and ovariectomized female mice. Nutr J 8, 11, doi:10.1186/1475-2891-8-11 (2009).
Hwang, L. L. et al. Sex differences in high-fat diet-induced obesity, metabolic alterations and learning, and synaptic plasticity deficits in mice. Obesity (Silver Spring) 18, 463-469, doi:10.1038/oby.2009.273 (2010).
Atamni, H. J., Mott, R., Soller, M. & Iraqi, F. A. High-fat-diet induced development of increased fasting glucose levels and impaired response to intraperitoneal glucose challenge in the collaborative cross mouse genetic reference population. BMC Genet 17, 10, doi:10.1186/s12863-015-0321-x (2016).
Dorfman, M. D. et al. Sex differences in microglial CX3CR1 signalling determine obesity susceptibility in mice. Nat Commun 8, 14556, doi:10.1038/ncomms14556 (2017).
Litwak, S. A. et al. Estradiol prevents fat accumulation and overcomes leptin resistance in female high-fat diet mice. Endocrinology 155, 4447-4460, doi:10.1210/en.2014-1342 (2014).
Olofsson, L. E., Pierce, A. A. & Xu, A. W. Functional requirement of AgRP and NPY neurons in ovarian cycle-dependent regulation of food intake. Proc Natl Acad Sci U S A 106, 15932-15937, doi:10.1073/pnas.0904747106 (2009).
Koebele, S. V. & Bimonte-Nelson, H. A. Modeling menopause: The utility of rodents in translational behavioral endocrinology research. Maturitas 87, 5-17, doi:10.1016/j.maturitas.2016.01.015 (2016).
Liu, T. et al. Fasting activation of AgRP neurons requires NMDA receptors and involves spinogenesis and increased excitatory tone. Neuron 73, 511-522, doi: 10.1016/j.neuron.2011.11.027 (2012).
Yang, Y., Atasoy, D., Su, H. H. & Sternson, S. M. Hunger states switch a flip-flop memory circuit via a synaptic AMPK-dependent positive feedback loop. Cell 146, 992-1003, doi:10.1016/j.cell.2011.07.039 (2011).
Moore, Y. E., Deeb, T. Z., Chadchankar, H., Brandon, N. J. & Moss, S. J. Potentiating KCC2 activity is sufficient to limit the onset and severity of seizures. Proc Natl Acad Sci U S A 115, 10166-10171, doi:10.1073/pnas.1810134115 (2018).
Moore, Y. E., Kelley, M. R., Brandon, N. J., Deeb, T. Z. & Moss, S. J. Seizing Control of KCC2: A New Therapeutic Target for Epilepsy. Trends Neurosci 40, 555-571, doi:10.1016/j.tins.2017.06.008 (2017).
Jaenisch, N., Witte, O. W. & Frahm, C. Downregulation of potassium chloride cotransporter KCC2 after transient focal cerebral ischemia. Stroke 41, e151-159, doi:10.1161/STROKEAHA.109.570424 (2010).
Dargaei, Z. et al. Restoring GABAergic inhibition rescues memory deficits in a Huntington's disease mouse model. Proc Natl Acad Sci U S A 115, E1618-E1626, doi:10.1073/pnas.1716871115 (2018).
Boulenguez, P. et al. Down-regulation of the potassium-chloride cotransporter KCC2 contributes to spasticity after spinal cord injury. Nat Med 16, 302-307, doi:10.1038/nm.2107 (2010).
Chen, B. et al. Reactivation of Dormant Relay Pathways in Injured Spinal Cord by KCC2 Manipulations. Cell 174, 521-535 e513, doi:10.1016/j.cell.2018.06.005 (2018).
Savardi, A., Borgogno, M., De Vivo, M. & Cancedda, L. Pharmacological tools to target NKCC1 in brain disorders. Trends Pharmacol Sci, doi:10.1016/j.tips.2021.09.005 (2021).
Ben-Ari, Y. & Cherubini, E. The GABA Polarity Shift and Bumetanide Treatment: Making Sense Requires Unbiased and Undogmatic Analysis. Cells 11, doi: 10.3390/cells11030396 (2022).
Khirug, S. et al. A single seizure episode leads to rapid functional activation of KCC2 in the neonatal rat hippocampus. J Neurosci 30, 12028-12035, doi:10.1523/JNEUROSCI.3154-10.2010 (2010).
Rossi, M. A. et al. Obesity remodels activity and transcriptional state of a lateral hypothalamic brake on feeding. Science 364, 1271-1274, doi:10.1126/science. aax1184 (2019).

1. A method of treating obesity in a subject by administering an agent that increases K-Cl co-transporter 2 protein (KCC2)'s function or expression.

2. The method of claim 1, wherein the agent is capable of increasing expression of Slc12a5 gene encoding KCC2.

3. The method of claim 2, wherein the agent is a CRISPR activation-Cas9 complex with gRNA that specifically targets Slc12a5 gene.

4. A pharmaceutical composition to treat obesity comprising a compound that increases K-Cl co-transporter 2 protein (KCC2)'s function or expression, and a pharmaceutically acceptable carrier.

5. A method of treating obesity in a subject by administering an agent that inhibits Na-K-Cl co-transporter 1 protein (NKCC1)'s function or expression.

6. The method of claim 5, wherein the agent is a chemical compound including bumetanide, azosemide, DIMAEB, BUM66, or a combination thereof.

7. The method of claim 5, wherein the agent is capable of inhibiting expression of Slc12a2 gene encoding NKCC1.

8. The method of claim 7, wherein the agent is a CRISPR-Cas9 complex comprising a gRNA that specifically targets Slc12a2 gene.

9. A pharmaceutical composition to treat obesity comprising a compound that inhibits Na-K-Cl co-transporter 1 protein (NKCC1)'s function or expression, and a pharmaceutically acceptable carrier.

10. The composition of claim 9, wherein the compound includes bumetanide, azosemide, DIMAEB, BUM66, or a combination thereof.

11. A pharmaceutical composition to treat obesity that comprises a first compound that increases K-Cl co-transporter 2 protein (KCC2)'s function or expression, a second compound that inhibits Na-K-Cl co-transporter 1 protein (NKCC1)'s function or expression, or combinations thereof, and a pharmaceutically acceptable carrier.

12. The composition of claim 11, wherein the second compound includes bumetanide, bumetanide, azosemide, DIMAEB, BUM66, or a combination thereof.

13. A method of treating obesity in a subject by administering the pharmaceutical composition of claim 12.
